(19) United States
(12) Patent Application Publication
(10) Pub. No.: US 2003/0013671 A1
(43) Pub. Date: Jan. 16, 2003

(54) Genomic DNA library

(76) Inventors: Junichi Mineno, Uji-shi (JP); Kiyozo Asada, Shiga-ken (JP); Ikunoshin Kato, Uji-shi (JP); Chikako Tanabe, Hiratsuka-shi (JP); Hiroki Sasaki, Tokyo-to (JP); Masaaki Terada, Tokyo-to (JP)

Correspondence Address: BIRCH STEWART KOLASCH & BIRCH, PO BOX 747, FALLS CHURCH, VA, 22040-0747, US

(21) Appl. No.: 09/989,420

(22) Filed: Nov. 21, 2001

(30) Foreign Application Priority Data
May 8, 2001 (JP) 2001-137858

(51) Int. Cl. C12Q 1/68; C07H 21/04; A61K 48/00; C12P 19/34

(52) U.S. Cl. 514/44; 435/6; 435/91.2; 536/23.1

(57) ABSTRACT

The present invention relates to a genomic DNA library substantially maintaining copy numbers of a set of genes or the sequences on a genome, an abundance ratio of the set of genes or sequences on the genome, and the polymorphic patterns substantially identical to that of the genomic DNA. The present invention also relates to a method of producing the above genomic DNA library. The genomic DNA library of the present invention is useful in analysis of genetic polymorphism; genetic diagnosis of a disease; preparation of DNA arrays; preparation of samples for searching open reading frames in analysis such as genome analysis; preservation of genes of endangered organisms; gene specimens; mutation analysis; nucleotide sequence analysis; analysis by hybridization methods such as Southern blot hybridization method, dot blot hybridization method, Northern blot hybridization method, macroarray hybridization methods using membrane and the like, or DNA microarray hybridization method.

### BACKGROUND OF THE INVENTION

1. Field of the Invention

The present invention relates to a genomic DNA library substantially maintaining copy numbers of a set of genes or the sequences on a genome, an abundance ratio of the set of genes or sequences on the genome, and polymorphic patterns substantially identical to that of the genomic DNA. The genomic DNA library is capable of giving a DNA substantially maintaining the copy numbers for a set of genes or the sequences on a genome, the abundance ratio of the set of genes or sequences on the genome, and the polymorphic patterns substantially identical to the genomic DNA, when the genomic DNA library is used as a template in nucleic acid amplification. The present invention also relates to a method of producing the above genomic DNA library.

2. Discussion of the Related Art

According to human genome analysis, it is thought that susceptibilities to diseases, possibilities of onset of diseases, individual constitutions, and the like can be determined, on the basis of genetic polymorphisms such as SNP. When statistical analysis is carried out on the basis of the typing of numerous genetic polymorphisms which have an effect on individual constitutions and the like, it is anticipated that the depletion of sources for the obtainment of the genomic DNA, for example, blood samples, and the like will be seriously problematic.

In addition, the preservation of DNA having various clinical information is thought to be essential in preparation for advances in genome sciences such as functional genome analysis; and responses to medicine, and the like.

Currently, means of DNA preservation include, for instance, a method for preserving a DNA by DNA amplification method, and the like. Such DNA amplification method includes a method of amplifying a restriction endonuclease-digested DNA fragment by appropriate means such as PCR; and DOP-PCR method, wherein a DNA fragment is amplified by an extension reaction using degenerated primers.

According to the method of amplifying a restriction endonuclease-digested DNA fragment by PCR, only a part of genomic DNA has been successfully amplified [M. S. H. KO et al., Nucleic Acids Res., 18, 4293-4294 (1990); H. Sasaki et al., Cancer Res., 54, 5821-5823 (1994); R. Lucito et al., Proc. Natl. Acad. Sci. USA, 95, 4487-4492 (1998)]. According to this method, the use of a various kinds of restriction endonucleases allows to cover 80 to 90% of the entire genomic DNA. However, since PCR tends to preferentially amplify short DNA fragments having 300 bp or less, the method of amplifying a restriction endonuclease-digested DNA fragment by PCR is faulty in that DNA is unevenly amplified, namely, certain size ranges of DNAs such as the short DNA fragments having 300 bp or less are preferentially amplified, thereby resulting in a DNA differing from the genomic DNA in terms of the polymorphisms, the copy number, and other properties.

Other available methods include the DOP-PCR , wherein a DNA fragment is amplified by an extension reaction using degenerated primers [H. Telenius et al., Genes Chromosomes Cancer, 4,257-263 (1992); Q. Huang et al., Genes Chromosomes Cancer, 28, 395-403 (2000); L. Zhang et al., Proc. Natl. Acad. Sci. USA, 89, 5847-5851 (1992); W. Dietmaier et al., AM. J. Pathol, 154, 83-95 (1999)]. However, as mentioned above, PCR tends to amplify shorter DNA fragments as the cycle number increases. In addition, it is difficult to amplify DNA by PCR in large scale, and the efficiency of amplification varies depending on the sequence of the genomic DNA, Therefore, there is faulty in that an unevenly constituted amplified product is obtained.

Therefore, there has been desired a demand for a genomic DNA library substantially maintaining the copy numbers for a set of genes or the sequences on a genome, the abundance ratio of the set of genes or sequences on the genome, and the polymorphic patterns substantially identical to the genomic DNA; and the development of a method for producing the genomic DNA library, capable of reflecting the quantitative ratio of the original copy numbers in the genomic DNA.

### SUMMARY OF THE INVENTION

A first object of the present invention is to provide a genomic DNA library substantially maintaining the copy numbers for a set of genes or the sequences on a genome, the abundance ratio of the set of genes or sequences on the genome, and the polymorphic patterns substantially identical to the genomic DNA. According to the genomic DNA library of the present invention, there can be obtained a DNA substantially maintaining the copy numbers for a set of genes or the sequences on a genome, the abundance ratio of the set of genes or sequences on the genome, and the polymorphic patterns substantially identical to the genomic DNA, when the genomic DNA library is used as a template in nucleic acid amplification.

A second object of the present invention is to provide a method for producing the genomic DNA library.

Specifically, the gist of the present invention follows:

[1] a genomic DNA library maintaining substantially copy numbers of a set of genes or sequences on a genomic DNA and an abundance ratio of the set of genes or sequences on the genomic DNA;

[2] the genomic DNA library according to the above item [1], which is obtained by carrying out a process comprising the steps of:

(1) subjecting a genomic DNA to DNA fragmentation means for generating a mixture of fragmented DNAs having distribution ratio of 1 to 5 as defined by the size ratio (distribution ratio) of the maximum size of fragmented DNA to the minimum size of fragmented DNA and having a size convergence rate of 80% or more, thereby giving a mixture of fragmented DNAs; and

(2) subjecting the mixture of fragmented DNAs obtained in step (1) to nucleic acid amplification, thereby producing DNAs corresponding to the mixture of fragmented DNAs;

[3] the genomic DNA library according to the above item [2], wherein the mixture of fragmented DNAs obtained in step (1) is a mixture of DNAs having distribution ratio of 1 to 5 as defined by the size ratio (distribution ratio) of the maximum size of fragmented DNA to the minimum size of fragmented DNA;

[4] the genomic DNA library according to the above item [2], wherein the mixture of fragmented DNAs obtained in step (1) is a mixture of DNAs having a size convergence rate of 80% or more;

[5] the genomic DNA library according to the above item [2], wherein the mixture of fragmented DNAs obtained in step (1) is a mixture of DNAs having an average size of from 0.8 kbp to 1.5 kbp;

[6] the genomic DNA library according to the above item [2], wherein the nucleic acid amplification is Polymerase Chain Reaction (PCR) method;

[7] a method for producing a genomic DNA library, comprising the steps of

(1) subjecting a genomic DNA to DNA fragmentation means for generating a mixture of fragmented DNAs having distribution ratio of 1 to 5 as defined by the size ratio (distribution ratio) of the maximum size of fragmented DNA to the minimum size of fragmented DNA and having a size convergence rate of 80% or more, thereby giving a mixture of fragmented DNAs; and

(2) subjecting the mixture of fragmented DNAs obtained in step (1) to nucleic acid amplification, thereby producing DNAs corresponding to the mixture of fragmented DNAs, to give a genomic DNA library maintaining substantially copy numbers of a set of genes or sequences on a genomic DNA and an abundance ratio of the set of genes or sequences on the genomic DNA;

[8] the method according to the above item [7], wherein the DNA fragmentation means is physical means;

[9] the method according to the above item [8], wherein the physical means is hydrodynamic point-sink shearing method;

[10] the method according to the above item [7], wherein the mixture of fragmented DNAs is a mixture of DNAs having distribution ratio of 1 to 5 as defined by the size ratio (distribution ratio) of the maximum size of fragmented DNA to the minimum size of fragmented DNA;

[11] the method according to the above item [7], wherein the mixture of fragmented DNAs is a mixture of DNAs having a size convergence rate of 80% or more;

[12] the method according to the above item [7], wherein the mixture of fragmented DNAs is a mixture of DNAs having an average size of from 0.8 kbp to 1.5 kbp;

[13] the method according to the above item [7], comprising the steps of:

(a) subjecting a genomic DNA to the DNA fragmentation means, thereby giving fragmented DNAs;

(b) ligating adapter DNA to the fragmented DNAs obtained in step (a), thereby giving DNA fragments; and

(c) carrying out nucleic acid amplification with the DNA fragments obtained in step (b) as a template and amplification primers, to give a genomic DNA library;

[14] the method according to the above item [13], wherein the DNA fragmentation means in step (a) is hydrodynamic point-sink shearing method;

[15] the method according to the above item [13], wherein the nucleic acid amplification in step (c) is Polymerase Chain Reaction (PCR) method;

[16] the method according to the above item [13], wherein the amplification primers used in the nucleic acid amplification in step (c) are primers selected from the group consisting of:

(i) oligonucleotides having a sequence complementary to the adapter DNA, and

(ii) oligonucleotides further comprising recognition sequences for restriction endonucleases, linker sequences and promoter sequence for RNA polymerase, in the sequence of the oligonucleotides of the above item (i);

[17] the method according to the above item [13], wherein the nucleic acid amplification in step (c) is carried out by using a DNA polymerase having a proofreading activity;

[18] the method according to the above item [17], wherein the DNA polymerase is a thermostable DNA polymerase;

[19] the method according to the above item [17], wherein the DNA polymerase is a mixture of a DNA polymerase having 3′→5′ exonuclease activity and a DNA polymerase having no 3′→5′ exonuclease activity;

[20] the method according to the above item [17], wherein the DNA polymerase is a mixture of at least two kinds of DNA polymerases each having 3′→5′ exonuclease activity;

[21] the method according to the above item [17], wherein the DNA polymerase is a mixture of α type DNA polymerase and non-α, non-pol I type DNA polymerase;

[22] a kit for producing a genomic DNA library, comprising the following amplification reagents (1) to (6):

(1) DNA ligase,

(2) enzymes for blunting a terminal of DNA,

(3) thermostable DNA polymerase,

(4) adapter DNA,

(5) reagents for PCR, and

(6) amplification primers selected from the group consisting of:

(i) oligonucleotides each having a sequence complementary to the adapter DNA, and

(ii) oligonucleotides further comprising at least one selected from the group consisting of recognition sequences for restriction endonucleases, linker sequences and promoter sequence for RNA polymerase, in the sequence of the oligonucleotides of the above item (i), and

comprising an instruction manual showing a procedure for carrying out the method of the above item [7] by using the amplification reagents, wherein the kit is used for production of the genomic DNA library of any one of the above item [1].

### BRIEF DESCRIPTION OF THE DRAWINGS

FIG. 1 shows the amplification pattern for microsatellite markers D4S1535, D3S1292, D2S337, and D3S3038 obtained by amplification with the genomic DNA library of the present invention as a template. In the figure, “O” represents amplified products derived from an original genomic DNA, and “A” represents amplified products derived from a genomic DNA library.

FIG. 2 shows the differences between copy numbers for the following genes: CAB1 gene, cyclin D1 gene, cyclin E gene and p16 gene, and the genetic characteristics in the genomic DNA library of the present invention. The p16 gene has a sequence with high GC contents.

FIG. 3 is the analytic results of nucleotide sequences of the amplified products each derived from thymine-DNA glycosilase gene of the genomic DNA library and original genomic DNA Panel A shows the result of the amplified products derived from the genomic DNA library of the present invention, and panel B shows the result of the amplified products derived from the genomic DNA.

FIG. 4 shows the electrophoretic patterns of the digested products of genomic DNA and the genomic DNA library of the present invention. In the figure, lane M represents pHY marker, lane 1 showing the digested DNA of the genomic DNA, lane 2 showing the digested DNA of the genomic DNA library obtained in Example 1, and lane 3 showing the digested DNA of the genomic DNA library obtained in Example 5.

FIG. 5 shows the electrophoretic patterns of the products obtained by digesting λDNA with EcoT14I, a mixture of EcoT14I and BglII, or HindIII.

### DETAILED DESCRIPTION OF THE INVENTION

The present invention is based on the surprising finding made by the inventors that a DNA obtained by subjecting a genomic DNA to DNA fragmentation means capable of exhibiting specific performances, thereby giving fragmented DNAs, and subjecting the fragmented DNAs to nucleic acid amplification, thereby producing DNAs corresponding to the fragmented DNAs, maintains copy numbers for a set of genes or sequences on a genome (e.g. a genomic DNA) and an abundance ratio of the set of genes or sequences on the genome.

One of the significant features of the genomic DNA library of the present invention resides in that the genomic DNA library substantially maintains the copy numbers of a set of genes or sequences on the genomic DNA and the abundance ratio of the set of genes or sequences on the genomic DNA. In addition, the genomic DNA library of the present invention is excellent in that the genomic DNA library reflects the quantitative ratio of the original copy numbers on the genome (e.g. genomic DNA). Therefore, when the genomic DNA library of the present invention is used, there can be reflected the genomic characteristics of the original genomic DNA, such as the copy numbers of a set of genes or sequences and the quantitative ratio of the original copy numbers of the genes or sequences. Concretely, according to the genomic DNA library of the present invention, there can be obtained a DNA substantially maintaining the copy numbers for a set of genes or the sequences on a genome, the abundance ratio of the set of genes or sequences on the genome, and the polymorphic patterns substantially identical to the genomic DNA, when the genomic DNA library is used as a template in nucleic acid amplification.

In addition, the genomic DNA library of the present invention is useful in analysis of genetic polymorphism; genetic diagnosis of a disease; preparation of DNA arrays; preparation of samples for searching open reading frames in analysis such as genome analysis; preservation of genes of endangered organisms; gene specimens; mutation analysis; nucleotide sequence analysis; analysis by hybridization methods such as Southern blot hybridization method, dot blot hybridization method, Northern blot hybridization method, macroarray hybridization methods using membrane and the like, or DNA microarray hybridization method.

In the present specification, the phrase “(to) maintain the copy numbers for a set of genes or sequences on the genome and the abundance ratio of the set of genes or sequences on the genome” refers to maintain preferably 85% or more, more preferably 90% or more, particularly preferably 95% or more of the copy numbers for a set of genes or sequences on the genome and the abundance ratio of the set of genes or sequences on the genome.

In addition, in the present specification, the genomic DNA library of the present invention is also referred to as “genomic DNA immortalized library”. Incidentally, the term “immortalize(d)” means to substantially maintain the copy numbers of a set of genes or sequences on the genomic DNA and the abundance ratio of the set of genes or sequences on the genomic DNA.

In the present specification, the term “fragmented DNA” as used herein refers to a mixture of several kinds of DNA fragments, unless otherwise stated.

In the present invention, depending on the purpose of use, the genomic DNA library of the present invention may be, for instance, a labeled genomic DNA library obtained by using labeled deoxynucleotide during its preparation, or may be a genomic DNA library ligated to an appropriate vector so as to facilitate gene cloning and the like, as occasion demands. Such genomic DNA libraries are also encompassed in the scope of the present invention.

In the present invention, whether or not the genomic DNA library of the present invention maintains the copy numbers for a set of genes or sequences on a genomic DNA, for example, is determined as described below. Concretely, whether or not the genome (the genomic DNA) and the genomic DNA library of the present invention are in the same level are evaluated by 1) hybridization analysis such as Southern blot hybridization analysis or slot blot hybridization analysis by using several kinds of labeled probes in the same amount (same specific radioactivity) corresponding to the same gene; and 2) comparison made between each of the signal intensities ascribed to the labeled probes hybridized. When the genome (the genomic DNA) and the genomic DNA library have the same copy numbers of a set of genes or sequence, their signal intensities should be substantially identical.

In addition, whether or not a genomic DNA library maintains the abundance ratio of a set of genes or sequences on a genome, for example, is determined as described below. Concretely, whether or not amplification patterns of the genome (the genomic DNA) and the genomic DNA library are identical are evaluated by 1′) subjecting the genomic DNA and the genomic DNA library to PCR using primers capable of specifically amplifying a set of genes or sequences, and 2′) subjecting each of the resulting amplified products to agarose gel electrophoresis. When the genome (the genomic DNA) and the genomic DNA library have the same abundance ratio of a set of genes or a sequence on the genome, their amplification patterns on electrophoresis should be substantially identical.

In the present invention, the copy number of the set of genes or sequence can be evaluated by determining the number of molecular for the set of genes or sequences on the original genomic DNA per a cell or the number of molecular for the set of the genes or the sequences on a DNA derived from the genomic DNA library per a transformed cell with the DNA genomic library of the present invention, by means of conventionally used method, such as one described in Saibokogaku Bessatsu “Shinpan Baiojikken Irasutoreiteddo, 3⁺, Hontouni Fueru PCR (New Edition, Bio-Experimental Illustrated, 3⁺, PCR for Real Amplification)” Dai 2 Han (2nd Edition), p. 141-186, published by Shujunsha.

The genomic DNA library of the present invention can be obtained by the steps of:

(A) subjecting a genomic DNA to DNA fragmentation means for preparing a mixture of fragmented DNAs having a distribution ratio of 1 to 5 as defined by the size ratio (distribution ratio) of the maximum size of fragmented DNA to the minimum size of fragmented DNA and having a size convergence rate of not less than 80%, thereby giving a mixture of fragmented DNAs; and

(B) subjecting the resulting mixture of fragmented DNAs obtained in step (A) to nucleic acid amplification, thereby producing DNAs corresponding to the mixture of fragmented DNA. Such a method for producing the genomic DNA library is also encompassed in the scope of the present invention.

The genomic DNA library maintaining the copy numbers for a set of genes or sequences on a genomic DNA and the abundance ratio of the set of genes or sequences on the genome is often difficult to be prepared by using a conventional technique for library preparation using many kinds of restriction endonucleases. However, according to the method for producing the genomic DNA library of the present invention, there are exhibited excellent effect that such a genomic DNA library can readily be produced.

One of significant features of the method for producing a genomic DNA library of the present invention resides in that the method comprises the steps of:

(A) subjecting a genomic DNA to DNA fragmentation means for generating a mixture of fragmented DNAs having has distribution ratio of 1 to 5 as defined by the size ratio (distribution ratio) of the maximum size of fragmented DNA to the minimum size of fragmented DNA and having a size convergence rate of not less than 80%, thereby giving a mixture of fragmented DNAs; and

(B) subjecting the resulting mixture of fragmented DNAs obtained in step (A) to nucleic acid amplification, thereby producing DNAs corresponding to the mixture of fragmented DNAs.

According to the method for producing a genomic DNA library of the present invention, since a fragmented DNA is obtained by treatment of a DNA with the DNA fragmentation means, there are exhibited some excellent effects such that imbalance in the amplification of fragments derived from shorter DNA fragments and uneven amplification can be suppressed. In addition, the method for producing a genomic DNA library of the present invention exhibits an excellent effect such that there can be produced in large scale a DNA maintaining the quantitative ratio of copy numbers of genes or sequences on the genomic DNA, the abundance ratio of a set of genes or sequences on the genomic DNA, the polymorphic patterns substantially identical to those of the genomic DNA.

In the method for producing genomic DNA library of the present invention, from the viewpoint of obtaining a genomic DNA library maintaining the quantitative ratio of copy numbers on a genomic DNA and the same polymorphism pattern as the genomic DNA, the fragmented DNA includes a mixture of DNAs having a distribution ratio of 5 or less as defined as the size ratio (distribution ratio) of the maximum size of fragmented DNA to the minimum size of fragmented DNA, concretely, a mixture of DNAs having a distribution ratio of 1 to 5.

The distribution ratio can be evaluated by, for example, the following steps:

[1] subjecting the fragmented DNA to a commonly used nucleic acid detection means;

[2] determining the sizes of the maximum size of DNA and the minimum size of DNA, respectively; and

[3] calculating the ratio of the maximum size of fragmented DNA to the minimum size of fragmented DNA.

The nucleic acid detection means in step [1] includes, for example, agarose gel electrophoresis, polyacrylamide gel electrophoresis, HPLC and the like.

In the step [2], the determination of size of DNA can be carried out by, for example, evaluating the mobility, mass, and the like of the fragmented DNA using a commonly used molecular weight marker or the like as a control.

In addition, when agarose gel electrophoresis or polyacrylamide gel electrophoresis is carried out in step [1], a band derived from DNA may optionally be visualized in step [2]. Useful means of visualizing a band derived from DNA include, but are not limited to, the intercalator type fluorescent dyes, for instance, ethidium bromide, SYBR-Green, SYBER-Gold, acridine, and Stain-All.

In addition, when HPLC is carried out in step [1], the step [1] can be carried out simultaneously with the step [2].

Furthermore, when HPLC is carried out, HPLC can also be carried out in the combination with a commonly used gel filtration method.

In the method for producing a genomic DNA library of the present invention, from the viewpoint of obtaining an amplified DNA maintaining the quantitative ratio of copy numbers for genes in the genomic DNA and the polymorphism patterns substantially identical to the genomic DNA, the fragmented DNA includes a mixture of DNAs having an average size of 0.8 kbp or more, preferably 0.8 kbp or more, and more preferably 0.5 kbp or more, and from the same viewpoint, the fragmented DNA is a mixture of DNAs having an average size of 2.5 kbp or less, preferably 1.5 kbp or less.

The average size of DNAs can be evaluated by subjecting the fragmented DNA to agarose gel electrophoresis, polyacrylamide gel electrophoresis, or the like, to thereby visualize the DNAs on the gel; reading the intensities of the bands on the gel by a densitometer, an image scanner, or the like, to thereby determine the DNA content for each size, and then calculating the average value on the basis of the amounts and sizes of DNAs.

In addition, in the method for producing a genomic DNA library of the present invention, from the viewpoint of obtaining an amplified DNA maintaining the quantitative ratio of copy numbers for genes in the genomic DNA and the polymorphism patterns substantially identical to the genomic DNA, the fragmented DNA includes a mixture of DNAs having a size convergence rate of 80% or more, preferably 85% or more, and more preferably 90% and more. The term “size convergence rate” as used herein refers to the percentage of 2-fold size distributions centering about the DNA fragment of the desired size to the entire DNA fragments prepared.

From the viewpoint of obtaining such a fragmented DNA, DNA fragmentation means includes a physical method. Concretely, the physical method includes, a physical method which can give a fragmented DNA having a distribution ratio of 1 to 5 as defined as the size ratio (distribution ratio) of the maximum size of fragmented DNA to the minimum size of fragmented DNA, and having a size convergence rate of 80% or more.

More concretely, the physical method includes the hydrodynamic point-sink shearing method [Peter J. Oefner et al., Nucleic Acids Res., 24, 3879-3886 (1996); Yvonne R. Thorstenson et al., Genome Research, 8, 848-855 (1998); U.S. Pat. No. 5,846,832], and the like. In the method for producing a genomic DNA library of the present invention, the hydrodynamic point-sink shearing method is preferred from the viewpoint of efficiently obtaining a fragmented DNA which meets the requirements for the distribution ratio, the size convergence rate, and the average size.

The term “hydrodynamic point-sink shearing method” as referred to herein is a technique for fragmentizing a DNA, comprising forcing a DNA solution through a tube having a tubular structure having a region with an abruptly narrowed width (also referred to as narrowed area),to accelerate the volume flow rate of the solution via the narrowed area in the tubular structure, thereby fragmentizing a DNA by the resistance thus generated. According to the hydrodynamic point-sink shearing method, a final DNA fragment having desired size can be obtained by adjusting the solution flow rate and the size of the narrowed area.

Incidentally, there are also encompassed in the scope of the present invention applications of other methods having functional abilities to give a fragmented DNA which meets the requirements for the distributions ratio, the size convergence rate, and the average size, in place of the physical method in the method for producing a genomic DNA library of the present invention.

The method for producing a genomic DNA library of the present invention includes, concretely a method comprising the following steps (a) to (c):

(a) subjecting a genomic DNA to the DNA fragmentation means, thereby giving fragmented DNAs;

(b) ligating adapter DNA to the fragmented DNAs obtained in step (a), thereby giving DNA fragments; and

(c) carrying out nucleic acid amplification with the DNA fragments obtained in step (b) as a template and amplification primers.

First, a genomic DNA is subjected to the DNA fragmentation means, thereby giving fragmented DNAs [referred to “step (a)”].

The genomic DNA which can be applied in the method for producing a genomic DNA library of the present invention may be any genomic DNAs. Such a genomic DNA can be prepared from biological samples such as cells and tissue, nucleic acid-containing samples such as those of viroids, viruses, bacteria, fungi, yeasts, plants, and animals, by a series of procedures, including commonly used methods, for instance, lytic treatment of cells, tissue, and the like by using detergents, sonication, shaking with stirring using glass beads or disruption by using French press; phenol extraction; various chromatographies such as ion exchange, gel filtration, and the like; gel electrophoresis; and density-gradient centrifugation.

The fragmented DNA obtained in step (a) may be subjected to appropriate procedures such as ethanol precipitation; concentration and/or desalting using microfilters and the like, as occasion demands.

Next, the fragmented DNA obtained in step (a) is then ligated with an adapter DNA having a sequence suitably used for a nucleic acid amplification reaction [referred to “step (b)”].

The adapter DNA may be any DNA, as long as it is suitable for specific amplification on the basis of a sequence existing in the adapter DNA in a nucleic acid amplification reaction. The adapter DNA includes a DNA having a sequence which is not present or is present at low frequencies in the genomic DNA to be amplified. In addition, the DNA is not particularly limited, as long as the DNA can be used for a nucleic acid amplification reaction, and has more preferably a sequence capable of preparing a primer suitable for PCR, more preferably LA (long and accurate)-PCR™.

Furthermore, the adapter DNA may have recognition sequences for the appropriate restriction endonucleases therein.

Regarding the form of the terminus of the adapter DNA, it is desirable, from the viewpoint of ligation reaction efficiency, that the terminus of the adapter DNA is blunt-ended. The form of the terminus include, for example, blunt ends resulting from treatment with restriction endonucleases such as SmaI, NruI, PvuII, EcoRV, and ScaI.

From the viewpoint of amplification specificity during PCR, it is desirable that the length of the adapter DNA is 20 bp or more.

The adapter DNA used for the method of the present invention may be synthesized by commonly used methods of synthesis, for instance, the phosphoramidite method, the phosphoric acid triester method, the H-phosphonate method, and the thiophosphonate method, thereby giving a desired nucleic acid sequence.

Regarding to the adapter DNA, a commercially available product can be used. The commercially available product includes, for instance, the EcoRI-NotI-BamHI adapter (manufactured by Takara Shuzo Co., Ltd.), and the like, but is not limited to those exemplified above.

Also, in the step (b), terminal repair treatment of the fragmented DNA obtained in step (a) may be carried out prior to ligation of the fragmented DNAs with adapter DNA, as occasion demands.

The terminal repair treatment may be carried out by using, for example, T4 DNA polymerase, Klenow Fragment, S1 nuclease, Mung Bean nuclease or the like.

In the ligation of the fragmented DNAs with adapter DNA, there can be used a normal-temperature DNA ligase, for instance, T4 DNA ligase or Escherichia coli DNA ligase. In addition, there can be used thermostable DNA ligase, hyperthermostable ligase, and the like.

The DNA fragment obtained in step (b) may be subjected to appropriate procedures such as ethanol precipitation; concentration and/or desalinization using microfilters and the like, as occasion demands.

Next, a nucleic acid amplification reaction is carried out with the DNA fragment obtained in step (b) as a template and amplification primers [referred to “step (c)”].

In the step (c), there can be used the DNA fragment in an amount suitable for the nucleic acid amplification reaction as a template.

As the nucleic acid amplification reaction, there can be selected commonly used nucleic acid amplification methods such as PCR method, without particular limitation. In particular, from the viewpoint of more satisfactorily maintaining the copy numbers for a set of genes or sequences on the genome and the abundance ratio of the set of genes or sequences on the genome, PCR method based on the LA technology, i.e., the LA-PCR™ method, is desirable. In addition, the PCR may be three-step DNA amplification comprising dissociation (denaturation) of a double-stranded template DNA into a single-stranded DNA, annealing of a primer to the single-stranded template DNA, and synthesis (extension) of a complementary strand from the primer, or two steps DNA amplification comprising the primer annealing and extension step at the same temperature and the denaturation step, which is so-called “shuttle PCR” [“PCR HOU SAIZENSEN (The Forefront of PCR Method)” in “Protein, Nucleic Acid and Enzyme”, extra issue, 41, 425-428 (1996)].

When a nucleic acid amplification reaction is carried out by PCR method, as DNA polymerases, any DNA polymerase can be used, as long as it is in common use for PCR methods. From the viewpoint of more satisfactorily maintaining the copy numbers for a set of genes or sequences on the genome and the abundance ratio of the set of genes or sequences on the genome, a DNA polymerase possessing proofreading activity (3′→5′ proofreading activity) is preferable.

In addition, from the viewpoint of suppressing uneven amplification due to secondary structure, differences in GC content, and the like on a genomic DNA, and from the viewpoint of more satisfactorily maintaining the copy numbers for a set of genes or sequences on the genome and the abundance ratio of the set of genes or sequences on the genome, it is more preferable that the DNA polymerase is a thermostable DNA polymerase. Furthermore, from the viewpoint of more satisfactorily maintaining the copy numbers for a set of genes or sequences on the genome and the abundance ratio of the set of genes or sequences on the genome, a DNA polymerase possessing a property suitable for LA (long and accurate)-PCR™ is more preferable.

Such DNA polymerases include α-type DNA polymerase, mixed type DNA polymerase and the like.

More concretely, the α-type DNA polymerase includes, for instance, α-type DNA polymerases derived from Pyrococcus furiosus (for instance, Pfu DNA polymerase and the like), a DNA polymerase derived from Thermococcus litralis (VENT DNA polymerase), a DNA polymerase derived from Pyrococcus sp. KOD1 (KOD DNA polymerase), a DNA polymerase derived from Pyrococcus sp. GB-D (DEEP VENT DNA polymerase) and the like. These α type DNA polymerases may be commercially available enzymes, and include, for instance, PyroBEST™ DNA polymerase (manufactured by Takara Shuzo Co., Ltd.), KOD™ DNA polymerase (manufactured by TOYOBO CO., LTD.), Vent™ DNA polymerase (manufactured by New England Biolab), Deep Vent™ DNA polymerase (manufactured by New England Biolab), Tli™ DNA polymerase (manufactured by Promega), Pwo™ TM DNA polymerase (manufactured by Boehringer), Pfu turbo™ DNA polymerase (manufactured by STRATAGENE) and the like.

The term “mixed type DNA polymerase” refers to a mixture of at least two kinds of DNA polymerases possessing different properties. The mixed type DNA polymerase includes a mixture of a DNA polymerase possessing 3′→5′ exonuclease activity and another DNA polymerase possessing substantially no 3′→5′ exonuclease activity; a mixture of at least two kinds of DNA polymerases each possessing 3′→5′ exonuclease activity; and a mixture of an α-type DNA polymerase and a non-α, non-Pol I type DNA polymerase. These mixed type DNA polymerases may be commercially available enzymes, and include, for instance, TaKaRa Ex Taq™ (manufactured by Takara Shuzo Co., Ltd.), Takara LA Taq™ (manufactured by Takara Shuzo Co., Ltd.), KOD dash™ (manufactured by TOYOBO CO., LTD., rTth DNA polymerase XL (manufactured by Perkin-Elmer), TaqPlus™ DNA polymerase (manufactured by STRATAGENE), Expand High Fidelity PCR system (manufactured by Roche Diagnostics), Advantage-HF DNA polymerase (manufactured by Clontech), and the like.

The term “another DNA polymerase possessing substantially no 3′→5′ exonuclease activity” as used herein encompasses naturally occurring DNA polymerases possessing no 3′→5′ exonuclease activity, or DNA polymerases exhibiting no 3′→5′ exonuclease activities resulting from artificial modification of the functional portion involved in the expression of 3′→5′ exonuclease activity.

The amplification primers used for the nucleic acid amplification reaction may be any primer having a nucleotide sequence substantially complementary to the adapter DNA, or having a nucleotide sequence present in the adapter DNA, as long as the primer is capable of extending the DNA strand from the 3′-end thereof.

The term “a nucleotide sequence substantially complementary to (adapter DNA)” as used herein means a nucleotide sequence capable of annealing to the adapter DNA under operating reaction conditions used, for instance, under the stringent conditions with Tm value as an index described in Lab Manual PCR [published by Takara Shuzo Co., Ltd, 13-17, (1996)], and then extending a DNA. Designing such a primer is known to those skilled in the art, and can be carried out in reference to, for example, Lab Manual PCR [published by Takara Shuzo Co., Ltd, 13-16, (1996)]. In addition, a commercially available primer construction software, for instance, OLIGO™ Primer Analysis software (manufactured by Takara Shuzo Co., Ltd.) can be used.

In addition, the amplification primers may be oligonucleotides having modification sequences not complementary to the nucleotide sequence of the adapter DNA, for instance, recognition sequences for appropriate restriction endonucleases, a linker sequence, or promoter sequence for RNA polymerase, added on the 5′-end side thereof, depending on the purpose of use of the genomic DNA library obtained by the method of the present invention and other factors.

The above promoter sequence for RNA polymerase includes, for instance, promoter sequence for SP6 RNA polymerase, promoter sequence for T7 RNA polymerase, promoter sequence for T3 RNA polymerase and the like.

It is desirable that the size of amplification primers used for the method of the present invention is 15 bases or more in length, preferably 20 bases or more in length, from the viewpoint of maintaining specificity for the adapter DNA, thereby better retaining the copy numbers for a set of genes or a sequence on a genome and the abundance ratio of the set of genes or sequence on the genome. In addition, it is desirable that the amplification primer is shorter than the full length of the adapter DNA, preferably 50 bases or less in length, more preferably 30 bases or less in length, from the viewpoint of amplification reaction efficiency. It is desired the sequence of the primer is substantially identical to the adapter DNA so as to allow the 3′-end side to anneal under stringent conditions.

Amplification primers include, concretely, primers selected from the group consisting of the following (i) and (ii):

(i) oligonucleotides each having a sequence complementary to the adapter DNA, and

(ii) oligonucleotides further comprising at least one selected from the group consisting of recognition sequences for restriction endonucleases, linker sequences and promoter sequence for RNA polymerase, in the sequence of the oligonucleotides of the item (i).

The amplification primers used for the method of the present invention are obtained by, for example, commonly used methods of synthesis, for instance, the phosphoamidite method, the phosphotriester method, the H-phosphonate method, and the thiophosphonate method, so as to have a given nucleotide sequence.

In the method for producing a genomic DNA library of the present invention, nucleic acid amplification reaction conditions may be appropriately set depending on the DNA polymerase, nucleic acid amplification method, and the like used.

The genomic DNA library thus obtained may be ligated to an appropriate vector which can be introduced to an appropriate host. In addition, depending on the purpose of use, a labeled deoxynucleotide may be used during the nucleic acid amplification reaction to yield a labeled genomic DNA library. As such vectors in cases where the host is Escherichia coli, for example, the plasmid vectors include pUC18, pUC19, pBlueScript, pET, pGEM and the like, and the phage vectors include lambda phage vectors such as λgt10 and λgt11.

According to the method for producing a genomic DNA library of the present invention, there can be obtained a DNA maintaining the copy numbers for a set of genes or sequences on the genome and the abundance ratio of the set of genes or sequences on the genome Therefore, the method of the present invention is useful in, for example, analysis of genetic polymorphism; genetic diagnosis of disease; preparation of DNA arrays; preparation of samples for searching open reading frames in genome analysis and the like; preservation of genes of rare or endangered organisms; mutation analysis; nucleotide sequence analysis, Southern blot analysis, and the like.

The method for producing a genomic DNA library of the present invention can be carried out more conveniently and rapidly by means of a kit for producing a genomic DNA, comprising the following amplification reagents (1) to (6):

(1) DNA ligase,

(2) enzymes capable of blunting a terminal of DNA,

(3) thermostable DNA polymerase,

(4) adapter DNA,

(5) reagents for PCR, and

(6) amplification primers selected from the group consisting of:

(i) oligonucleotides each having a sequence complementary to the adapter DNA, and

(ii) oligonucleotides further comprising at least one selected from the group consisting of recognition sequences for restriction endonucleases, linker sequences and promoter sequence for RNA polymerase, in the sequence of the oligonucleotides of the item (i), and

comprising an instruction manual showing a procedure for carrying out the method for producing a genomic DNA library of the present invention by using the amplification reagents, wherein the kit is used for production of the genomic DNA library of the present invention. Such DNA amplification kits are also encompassed in the scope of the present invention.

The DNA ligase of the item (1) includes the same DNA ligases as those exemplified for the method for producing a genomic DNA library of the present invention.

The enzyme capable of blunting a terminal of DNA of the item (2) includes various enzymes mentioned for end repair treatment in the method for producing a genomic DNA library of the present invention.

The thermostable DNA polymerase of the item (3) and the adapter DNA (4) above are the same ones as exemplified the DNA polymerases and adapter DNAs in the method for producing a genomic DNA library of the present invention.

The reagents for PCR of the item (5) include dNTP mixture, magnesium chloride, and reaction buffers suitable for the thermostable DNA polymerase of the item (3).

The amplification primers of the item (6) include the same oligonucleotides as those exemplified for the method for producing a genomic DNA library of the present invention.

The instruction manual provides directions for procedures of carrying out the method for producing a genomic DNA library of the present invention using the kit, showing that by carrying out the method for producing a genomic DNA library of the present invention in accordance with the instructed procedures by using the amplification reagents, a genomic DNA library maintaining the copy numbers for a set of genes or sequences on the genomic DNA and the abundance ratio of the set of genes or sequences on the genome is obtained.

The instruction manual is a printed matter describing how to use the kit, for instance, the method of preparing reagents for making the aforementioned library, recommended reaction conditions, and the like, and includes those appearing on labels attached to the kit, packages housing the kit, and the like, as well as handling brochures in a pamphlet or leaflet form.

Furthermore, the information disclosed and provided via computer-readable recording media such as FD, MO, CD-ROM and DVD-ROM is also encompassed in the instruction manual. Kits accompanied by an instruction manual providing directions for operating conditions for the aforementioned amplification reagents in the making of library preparation reagents, or kits wherein the method of the present invention is disclosed and provided via an electronic medium such as the internet, are also encompassed in the scope of the kit of the present invention.

The kit for producing a genomic DNA library of the present invention may further comprise various reagents such as sterilized water and TE buffer.

### EXAMPLES

### Example 1

(1) Fragmentation of Genomic DNA

Each of genomic DNA for the gastric cancer cell line MKN74 and genomic DNA for the esophageal squamous cell cancer cell line TE6 was extracted by a commonly used nucleic acid extraction method (SDS-phenol.chloroform method). Two micrograms of each genomic DNA obtained was dissolved in 200 μl of TE buffer [composition: 10 mM Tris-HCl, 1 mM EDTA (pH 8.0)], to give genomic DNA solution. The resulting DNA solution was fragmented (shearing speed: 5) by using the random DNA fragmentation apparatus HydroShear™ (manufactured by Genomic Instrumentation Service), and 190 μl of fragmented product was recovered. Ten microliters of TE buffer was added to the recovered fragmented product to give 200 μl of DNA solution. To the resulting DNA solution, 200 μl of water-saturated phenol solution was added, with stirring, and the mixture solution was then centrifuged to recover supernatant. Two-hundred microliters of chloroform was added to the recovered supernatant, with stirring, and the resulting mixture solution was then centrifuged to recover supernatant. The supernatant obtained was further subjected to isopropanol precipitation. The precipitate obtained was rinsed with 70% ethanol and then dried, thereby giving pellets. The pellets obtained were dissolved in 20 μl of TE buffer to give a fragmented DNA solution.

(2) Blunting Treatment-1 of the Fragmented DNA

Ten microliters of BAL31 nuclease reaction buffer [composition of 5×concentrated buffer: 100 mM Tris-HCl (pH 8.0), 3 M NaCl, 60 mM CaCl₂, 60 mM MgCl₂ and 5 mM EDTA] and 35 μl of sterilized water were added to 5 μl of the fragmented DNA solution obtained in item (1) above, and the mixture was incubated at 70° C. for 5 minutes, and then incubated at 30° C. for 5 minutes. Thereafter, to the resulting product, 1.5 U of BAL31 nuclease (manufactured by Takara Shuzo Co., Ltd.) was added, and the resulting mixture was incubated at 30° C. for 1 minute. To the reaction mixture obtained, 50 μl of TE buffer was added. To the solution obtained, 100 μl of water-saturated phenol solution was added, with stirring. The resulting mixture was then centrifuged to recover supernatant. One-hundred microliters of chloroform was added to the recovered supernatant, with stirring, and the resulting mixture solution was then centrifuged to recover supernatant. The supernatant obtained was subjected to ethanol precipitation, and the precipitate was rinsed with 70% ethanol and then dried, thereby giving pellets. The pellets obtained were dissolved in 9 μl of sterilized water to give a BAL nuclease-treated DNA solution.

(3) Blunting Treatment-2 of the Fragmented DNA

Nine microliters of the BAL31 nuclease-treated DNA solution obtained in item (2) above was subjected to DNA blunting treatment by using a DNA Blunting Kit (manufactured by Takara Shuzo Co., Ltd.). Ninety microliters of TE buffer was added to 10 μl of the reaction mixture obtained. One-hundred microliters of a water-saturated phenol solution was added to the solution obtained, with stirring, and the solution was then centrifuged to recover supernatant. One-hundred microliters of chloroform was added to the supernatant obtained, with stirring, and the resulting mixture was then centrifuged to recover supernatant. The supernatant obtained was subjected to isopropanol precipitation. The precipitate obtained was rinsed with 70% ethanol and then dried, thereby giving pellets. The pellets obtained were dissolved in 25 μl of TE buffer.

(4) Adapter Ligation

To 1 μl (equivalent to about 0.015 μg) of the blunt-ended DNA obtained in item (3) above, 500 pmol of the EcoRI-NotI-BamHI adapter (manufactured by Takara Shuzo Co., Ltd.), 2 μl of 10×ligation buffer (manufactured by Takara Shuzo Co., Ltd.), 350 U of T4 DNA ligase (manufactured by Takara Shuzo Co., Ltd.), and 1 μl of 10 mM ATP were added, and sterilized water was added thereto to make up a total volume of 20 μl. Thereafter, the solution obtained was incubated at 15° C. for 16 hours to ligate adapter to the blunt-ended DNA (adapter ligation).

(5) 1st PCR

To 1 μl of the DNA solution after adapter ligation in item (4) above, 100 pmol of the ER1 primer of SEQ ID NO: 1, 10 μl of 2.5 mM dNTP mix, 5 U of TaKaRa Ex Taq™ DNA polymerase (manufactured by Takara Shuzo Co., Ltd.), and 10 μl of 10×PCR buffer were added, and sterilized water was added thereto to make up a total liquid volume of 100 μl. A reaction tube containing the solution obtained was set on the TaKaRa PCR Thermal Cycler MP (manufactured by Takara Shuzo Co., Ltd.), and PCR was carried out under the following conditions of:

incubating at 95° C. for 5 minutes,

carrying out 15 cycles of reaction, wherein one cycle of reaction is 95° C., 1 minute −72° C., 3 minutes; and

incubating at 72° C. for 10 minutes.

(6) 2nd PCR

To 20 μl of the reaction mixture after the 1st PCR in item (5) above, 100 pmol of the ER1 primer, 10 μl of 2.5 mM dNTP mix, 5 U of TaKaRa Ex Taq™ DNA polymerase, and 10 μl of 10×PCR buffer were added, and sterilized water was added thereto to make up a total liquid volume of 100 μl. A reaction tube containing the solution obtained was set on the TaKaRa PCR Thermal Cycler MP, and PCR was carried out under the following conditions of:

incubating at 95° C. for 5 minutes;

carrying out 5 cycles of reaction, wherein one cycle of reaction is 95°, 1 minute −72° C., 3 minutes; and

incubating at 72° C. for 10 minutes.

After the 2nd PCR, 100 μl of the reaction mixture obtained was subjected to isopropanol precipitation, and the resulting precipitate was rinsed with 70% ethanol and dried. The pellets obtained were dissolved in 20 μl of TE buffer to give a genomic DNA library.

(7) Confirmation of Amplification of APC Gene

Using the above-mentioned genomic DNA library obtained in item (6) above as a template, 9 kinds of DNA fragments of the antioncogene APC were amplified by PCR method with primers each having any one of the nucleotide sequences of SEQ ID NOs: 2 to 19. The combinations of primers and the deduced lengths of amplified fragments (shown as “deduced length of amplified fragment” in the table) are shown in Table 1.

TABLE 1

| Combination of | Deduced Length of |
|---|---|
| Primers | Amplified Fragment |
| (SEQ ID NO:) | (bp) |
| 2, 3 | 303 |
| 4, 5 | 295 |
| 6, 7 | 301 |
| 8, 9 | 327 |
| 10, 11 | 399 |
| 12, 13 | 649 |
| 14, 15 | 1038 |
| 16, 17 | 1372 |
| 18, 19 | 1408 |

PCR was carried out under the following conditions of:

carrying out 40 cycles of reaction, wherein one cycle of reaction is 94° C., 10 seconds −56° C., 20 seconds −72° C., 30 seconds; and

incubating at 72° C. for 3 minutes.

Five microliters of the reaction mixture obtained was subjected to agarose electrophoresis to confirm an amplified product. As a result, a DNA fragment having a deduced size was found. Therefore, it is found that the genomic DNA library thus obtained (the genomic DNA immortalized library) maintains the sequence of the original genomic DNA. It is found that the genomic DNA immortalized library is obtained by the method of the present invention without impairing the sequence of the original genomic DNA. Furthermore, according to the method of the present invention, 10 mg or more of a genomic DNA immortalized library could be prepared from 1 μg of the original genomic DNA.

### Example 2

The method for producing a genomic DNA immortalized library described in Example 1 was studied.

(1) Study on Method for Producing Genomic DNA Immortalized Library of the Present Invention with Microsatellite Marker as Control

Regarding the genomic DNA immortalized library obtained by the production method of the present invention, it was examined whether or not non-uniformity on the amount of the fragment obtained having a specific size is caused. A genomic DNA was prepared from specimen from esophageal mucosa obtained with informed consent by a conventional method. Using the genomic DNA obtained, a genomic DNA immortalized library was prepared according to the method described in Example 1.

Using the genomic DNA immortalized library obtained as a template, each of microsatellite markers D4S1535, D3S1292, D2S337, D3S3038, D2S123, D5S346, D17S250 and BAT23 was amplified by PCR with Human Map Pair [Human Screening Set. Ver. 9a labeled (ABI dye; manufactured by Research Genetics)]. The amplified products obtained were analyzed using the genetic analyzer ABI PRISM™ 310 (manufactured by PE Biosystems).

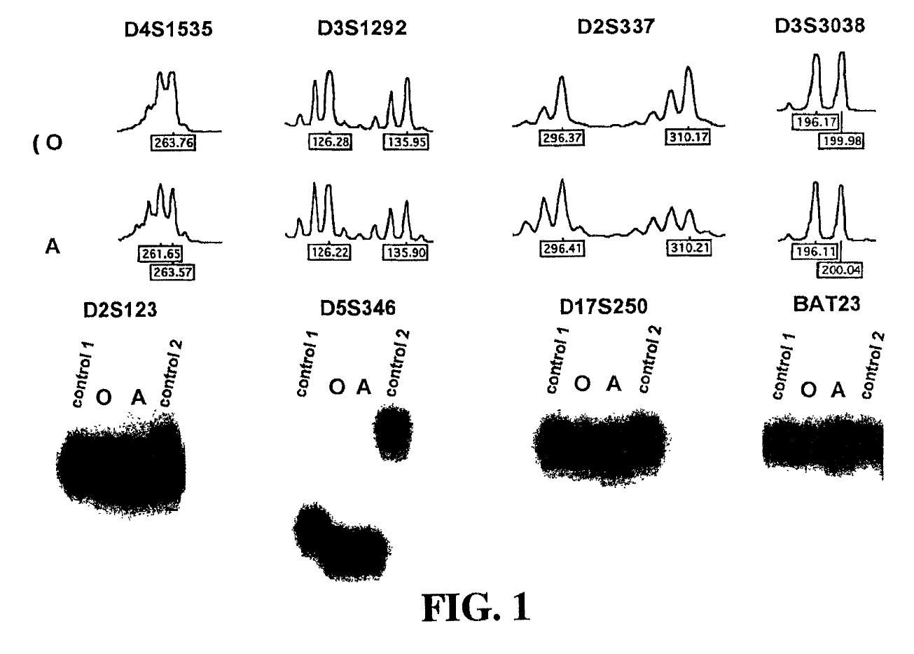

Further, each PCR amplified product was labeled with ³²P using the Random Primer DNA Labeling Kit (manufactured by Takara Shuzo Co., Ltd.). The resulting product was electrophoresed on 6% denatured polyacrylamide gel. The results are shown in FIG. 1. In FIG. 1, O is an electrophoretogram of amplified products from the original genomic DNA, and A is an electrophoretogram of amplified products from the genomic DNA immortalized library of the present invention.

As shown in FIG. 1, since the electrophoretic patterns of the amplified products from the original genomic DNA and those of the amplified products from genomic DNA immortalized library obtained by the method of the present invention were confirmed to have the same analytical patterns, it is found that the genomic DNA immortalized library of the present invention has the same patterns as the original genomic DNA. In other words, it is found that the abundance ratio of a set of genes on the genome is kept in the genomic DNA immortalized library of the present invention. In addition, it is confirmed that the genomic DNA immortalized library is obtained by the method of the present invention, with keeping the abundance ratio of a set of genes on the genome.

(2) Genomic DNA Immortalized Library Obtained by the Method of the Present Invention

The copy numbers for each gene in the genomic DNA immortalized library obtained by the production method of the present invention and the original template genomic DNA were studied.

A genomic DNA was prepared by a conventional method from each of a specimen (C1) from placenta, a specimen (C2) from normal esophageal mucosa and an esophageal cancer cell line (TE6) obtained with informed consent, and a genomic DNA immortalized library was prepared by the method of Example 1.

Four-hundred and fifty nanograms, 150 ng or 50 ng of DNA from the genomic DNA immortalized library obtained was subjected to slot-blotting to a membrane filter Hybond™-N⁺ (manufactured by Amersham-Pharmacia) using a convertible filtering apparatus (manufactured by Lifetec) to give a blot membrane. In addition, as a control, 10 μg of each of the above-mentioned three kinds of genomic DNA was digested with EcoRI 50U (manufactured by Takara Shuzo Co., Ltd.), and the product obtained was electrophoresed on 1% agarose gel. Thereafter, DNA on the 1% agarose was transferred to the membrane filter Hybond™-N⁺ (manufactured by Amersham-Pharmacia) to give a control membrane.

As a gene to be analyzed for hybridization, there were selected CAB1 gene (GenBank accession number: D38255), cyclin D1 gene (GenBank accession number: M64349), cyclin E1 gene (GenBank accession number: M73812), and p16 gene (GenBank accession number: L27211).

First, PCR was carded out using a primer pair in which each primer has the nucleotide sequences of SEQ ID NOs: 20 to 27. After the amplified fragment obtained was purified, the purified product was ligated to plasmid vector pT7Blue-T (manufactured by Novagen) by a conventional method. The recombinant plasmid obtained was used as a probe for hybridization analysis.

Concretely, 20 ng of the recombinant plasmid in which a full length of any one of genes was cloned was labeled with ³²P using Random Primer DNA Labeling Kit (manufactured by Takara Shuzo Co., Ltd.) in accordance with the instruction attached thereto and used.

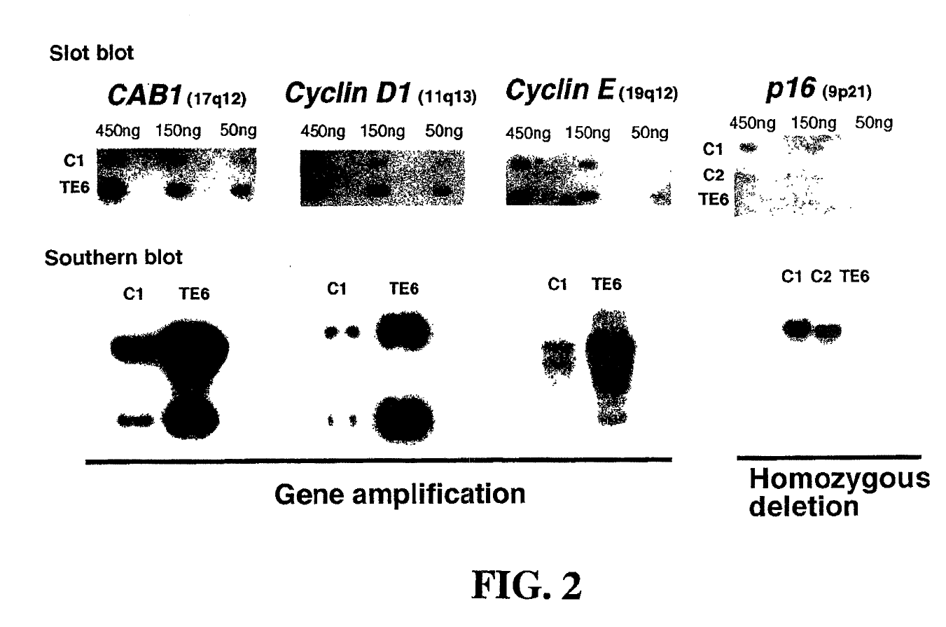

The probe was dissolved in a solution having the following composition of 50% formamide (manufactured by Nacalai Tesque), 5×SSC [composition of 1×SSC: 0.15 M NaCl, 0.015 M sodium citrate, (pH 7.0)], 5×Denhardt's solution, 5 mM EDTA, 0.1% SDS, 10% dextran sulfate, 100 mg/ml denatured salmon sperm DNA, to give a probe solution. Thereafter, hybridization was carried out by incubating each of the above-mentioned blot membrane, the control membrane and the above-mentioned probe solution at 42° C. for 16 hours. The membrane obtained was washed twice with a solution containing 0.1×SSC and 0.1% SDS at room temperature, and then washed twice at 65° C. After washing, the membrane filter was exposed to an XAR film (manufactured by Kodak), and the film was sensitized to obtain an autoradiogram. FIG. 2 shows the analytical results of slot blotting and Southern blotting.

As shown in FIG. 2, it is found that the genomic DNA immortalized library of the present invention maintains the difference of the copy number in the original genomic DNA. Namely, it is confirmed that the genomic DNA immortalized library is obtained by the method for producing a genomic DNA immortalized library of the present invention, with keeping the difference of the copy number in the original genomic DNA. Also, it is found that p16 with homozylous deletion in TE6 is also not detected in the genomic DNA immortalized library of the present invention.

### Example 3

(1) Preparation of Template Genomic DNA Fragment

A genomic DNA was prepared from a blood specimen obtained with informed consent by a conventional method. Two micrograms of the genomic DNA obtained was dissolved in 200 μl of TE buffer to give a DNA solution. The DNA solution obtained was subjected to fragmentation of the genomic DNA, blunting treatment of the fragment obtained, and thereafter adapter ligation treatment in the same manner as the method described in Example 1.

(2) 1st Shuttle PCR

To the genomic DNA fragment prepared in item (1) above as a template, 100 pmol of the ER1 primer, 10 μl of 2.5 mM dNTP mix, 5 U of TaKaRa Ex Taq™ DNA polymerase (manufactured by Takara Shuzo Co., Ltd.), and 10 μl of 10×PCR buffer were added, and sterilized water was added thereto to make up a total liquid volume of 100 μl. A reaction tube containing the solution obtained was set on the TaKaRa PCR Thermal Cycler MP, and PCR was carried out under the following conditions of:

incubating at 94° C. for 2 minutes; and

carrying out 15 cycles of reaction, wherein one cycle of reaction is 94° C., 15 seconds −68° C., 2 minutes.

(3) 2nd Shuttle PCR

To 20 μl of the 1st shuttle PCR solution obtained in item (2) above, 100 pmol of the ER1 primer, 10 μl of 2.5 mM dNTP mix, 5 U of TaKaRa Ex Taq™ DNA polymerase, and 10 μl of 10×PCR buffer were added, and sterilized water was added thereto to make up a total liquid volume of 100 μl. A reaction tube containing the solution obtained was set on the TaKaRa PCR Thermal Cycler MP, and PCR was carried out under the following conditions of:

incubating at 94° C. for 2 minutes; and

carrying out 5 cycles of reaction, wherein one cycle of reaction is 94° C., 15 seconds −68° C., 2 minutes,

After the 2nd PCR, the amount of the genomic DNA immortalized library obtained was about 10 μg per 1 ng of the template genomic DNA in the 1st PCR.

(4) Studies on the Genomic DNA Immortalized Library Prepared by the Production Method of the Present Invention

With the genomic DNA immortalized library prepared in item (3) above as a template, analysis of exons of gene was carried out.

As subject genes, there were selected BRCA1 gene (GenBank accession number: U14680) and thymine-DNA glycosylase gene (TDG: thymine-DNA glycosylase, GenBank accession number: NM 003211). The nucleotide sequences of the primers for amplifying these genes are shown in SEQ ID NOs: 28 to 61, respectively. As a control, PCR was also carried out with the genomic DNA prepared in item (1) above as a template.

PCR was carried out under the following conditions of:

incubating at 95° C. for 2 minutes; and

carrying out 30 cycles of reaction, wherein one cycle of reaction is 95° C., 15 seconds −61° C., 30 seconds −68° C., 30 seconds.

After the PCR, 5 μl of the reaction mixture obtained was subjected to 1.5% agarose electrophoresis. As a result, the amplification patterns ascribed to the genomic DNA immortalized library prepared by the method of the present invention and the amplification pattern ascribed to the genomic DNA were the same. In other words, in the genomic DNA immortalized library, the abundance ratio of a set of genes on the genome is kept.

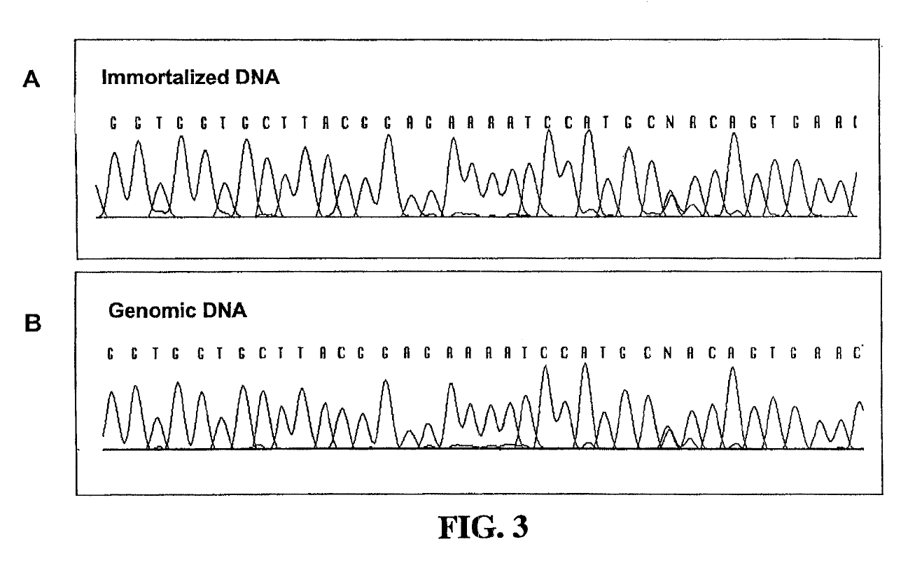

Furthermore, regarding the above-mentioned TDG gene, each of the nucleotide sequences of the amplified fragment ascribed to the genomic DNA immortalized library prepared by the method of the present invention and the amplified fragment ascribed to the genomic DNA was analyzed. The results are shown in FIG. 3. FIG. 3 shows the results of the analysis of the nucleotide sequence of the thymine-DNA glycosylase gene fragment amplified by PCR using the primer pair in which each primer has any one of sequences of SEQ ID NOs: 58 to 59.

As shown in FIG. 3, the nucleotide sequence patterns were the same between the amplified fragment ascribed to the genomic DNA immortalized library and the amplified fragment ascribed to the genomic DNA. It is found from above that the genomic DNA immortalized library of the present invention is also identical to the genomic DNA on the nucleotide sequence level. Namely, it is found that the genomic DNA immortalized library is obtained by the production method, with keeping the nucleotide sequence patterns on the genome.

(5) SNP Analysis Using the Library Prepared by the Method of the Present Invention

SNP patterns for the amplified fragments obtained in item (4) above were studied.

As a subject gene, TDG gene was selected. PCR amplification was carried out for each of the genomic DNA immortalized library prepared by the method of the present invention and the genomic DNA, using the primer pair in which each primer has the nucleotide sequences of SEQ ID NOs: 60 to 61. Comparison of the nucleotide sequences of the amplified fragments obtained revealed that both the genomic DNA immortalized library and the genomic DNA had the same SNP patterns. Therefore, it is found that the genomic DNA immortalized library maintains the same SNP patterns as those on the genome. Namely, it is found that the genomic DNA immortalized library is obtained by the production method of the present invention, with keeping the same SNP patterns on the genome.

### Example 4

Point Mutation of the p53 Gene

As a genomic DNA and a genomic DNA immortalized library prepared by the method of the present invention, the genomic DNA and the genomic DNA immortalized library each prepared by the method described in Example 1 were used. Further, as a control, the blood-derived genomic DNA described in Example 3 was used, As primers, oligonucleotides having the nucleotide sequences of SEQ ID NOs: 62 to 63 were used.

PCR was carried out under the following conditions of:

incubating at 95° C. for 3 minutes;

carrying out 35 cycles of reaction, wherein one cycle of reaction is 95° C., 45 seconds −55° C., 45 seconds −72° C., 1 minute; and

incubating at 72° C. for 10 minutes.

After the PCR, the nucleotide sequences of the amplified fragments obtained were analyzed by a conventional method. As a result, the nucleotide sequence of codon 248th of p53 gene from a normal individual was identified as CGG, whereas the nucleotide sequence of codon 248th of p53 gene from the amplified fragment ascribed to the genomic DNA immortalized library prepared by the method of the present invention and the nucleotide sequence of codon 248th of p53 gene from the amplified fragment ascribed to the genomic DNA was identified as CAG, so that the existence of a point mutation could be confirmed. In other words, according to the production method of the present invention, it is found that a library possessing the same genetic characteristics as the original genomic DNA can be obtained.

### Example 5

(1) Fragmentation of Genomic DNA

Two micrograms of Human Genomic DNA (manufactured by Clontech) was dissolved in 200 μl of TE buffer to give DNA solution. The DNA solution obtained was fragmented in the same manner as in item (1) of Example 1 to give a fragmented DNA solution.

(2) Blunting Treatment-1 of the Fragmented DNA

To 50 μl of the fragmented DNA solution obtained in item (1) above, 12.5 μl of a 5-fold concentrated reaction buffer for BAL31 nuclease was added. The solution obtained was incubated at 70° C. for 5 minutes and then incubated at 30° C. for 5 minutes. Thereafter, 1.5 U of BAL31 nuclease was added to the solution obtained, and the mixture was incubated at 30° C. for 1 minute. To the reaction mixture obtained, 50 μl of 50 mM EDTA solution was added to give a BAL31 nuclease-treated DNA solution.

(3) Blunting Treatment-2 of the Fragmented DNA

The amount 112.5 μl of the BAL31 nuclease-treated DNA solution was purified using Microcon™-100 (manufactured by Takara Shuzo Co., Ltd.). Thereafter, 5 μl of a 10-fold concentrated blunting buffer (manufactured by Takara Shuzo Co., Ltd.) and 45 μl of sterilized water were added to the purified solution obtained. To 40 μl of the solution obtained, 5 U of PyroBEST™ DNA polymerase (manufactured by Takara Shuzo Co., Ltd.) was added. The resulting mixture was incubated at 74° C. for 10 minutes, and then cooled to 4° C.

(4) Adapter Ligation

To 2 μl (equivalent to about 0.02 μg) of the blunt-ended DNA obtained in item (3) above, 500 pmol of the EcoRI-NotI-BamHI adapter (manufactured by Takara Shuzo Co., Ltd.) was added. Using the solution obtained and DNA Ligation Kit Ver. 2 (manufactured by Takara Shuzo Co., Ltd.), a ligation solution was prepared. The above ligation solution was incubated at 16° C. for 30 minutes to give an adapter ligated DNA solution.

(5) 1st PCR

To 1 μl of the adapter ligated DNA solution obtained in item (4) above, 100 pmol of the ER1 primer which was used in Example 1, 10 μl of 2.5 mM dNTP mix, 5 U of TaKaRa Ex Taq™ DNA polymerase, and 10 μl of 10×PCR buffer which were used in Example 1 were added, and sterilized water was added thereto to make up a total liquid volume of 100 μl. A reaction tube containing the solution obtained was set on the TaKaRa PCR Thermal Cycler MP, and PCR was carried out under the following conditions of:

incubating at 95° C. for 5 minutes;

carrying out 15 cycles of reaction, wherein one cycle of reaction is 95° C., 1 minute −72° C., 3 minutes; and

incubating at 72° C. for 10 minutes.

(6) 2nd PCR

To 20 μl of the solution after the 1st PCR, 100 pmol of the ER1 primer, 10 μl of 2.5 mM dNTP mix, 5 U of TaKaRa Ex Taq™ DNA polymerase, and 10 μl of 10×PCR buffer were added, and sterilized water was added thereto to make up a total liquid volume of 100 μl. A reaction tube containing the reaction mixture was set on the TaKaRa PCR Thermal Cycler MP, and PCR was carried out under the following conditions of:

incubating at 95° C. for 5 minutes;

carrying out 5 cycles of reaction, wherein one cycle of reaction is 95° C., 1 minute −72° C., 3 minutes; and

incubating at 72° C. for 10 minutes, By the above reaction, a genomic DNA immortalized library was obtained.

(7) Confirmation of Genome Immortalization

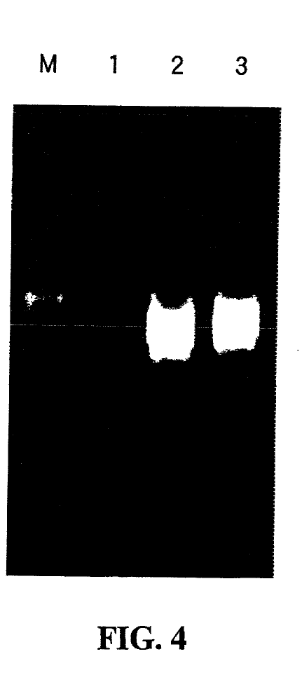

The electrophoretogram of 5 μl of the fragmented DNA obtained in item (1) above, 5 μl of the genomic DNA immortalized library obtained in Example 1, and 5 μl of the genomic DNA immortalized library obtained in item (6) above is shown in FIG. 4. In FIG. 4, lane M shows an electrophoretogram for the pHY molecular weight marker, lane 1 shows an electrophoretogram for the fragmented DNA, lane 2 shows an electrophoretogram for the library prepared in Example 1, and lane 3 shows an electrophoretogram for the library prepared in this Example.

FIG. 4 is an electrophoretogram. As shown in FIG. 4, it is found that the genomic DNA immortalized libraries of Examples 1 and 5 both gave the same electrophoretic patterns as the genomic DNA.

PCR was carried out by using the genomic DNA and the genomic DNA immortalized library obtained in item (6) above as templates. As genes to be analyzed were selected three kinds of genes: human CD59 gene (GenBank accession number: M34671), human DNA topoisomerase I gene (GenBank accession number: J03250), and human ATP-dependent DNA helicase II gene (GenBank accession number: M32865). The nucleotide sequences of the primers used are shown in SEQ ID NOs: 64 to 69. By using the combinations of the above-mentioned primers, it was deduced that an amplified fragment having a size of 770 bp for the human CD59 gene, an amplified fragment having a size of 791 bp for the human DNA topoisomerase I gene, and an amplified fragment having a size of 692 bp for human ATP-dependent DNA helicase II gene.

PCR was carried out under the following conditions of:

carrying out 40 cycles of reaction, wherein one cycle of reaction is 94° C., 10 seconds −56° C., 20 seconds −72° C., 30 seconds; and

incubating at 72° C. for 3 minutes.

After termination of PCR, when the DNA nucleotide sequences were confirmed by a conventional method using 5 μl of the reaction mixture, the desired DNA sequences were obtained in all amplified fragments.

It is therefore found that the genomic DNA obtained by this method is amplified without impairing the nucleotide sequence of the original genomic DNA. Therefore, it is found that the genomic DNA immortalized library of the present invention has the same genetic pattern as the original genomic DNA. Namely, it is found that a genomic DNA immortalized library can be constructed according to the method of the present invention.

### Example 6

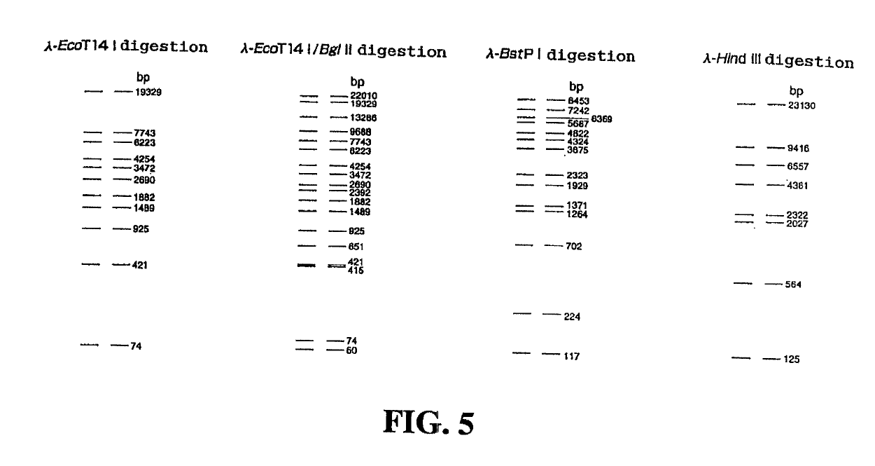

A λ phage DNA was isolated by a commonly used method. Thereafter, the λDNA obtained used as a substrate was digested by various kinds of restriction endonucleases: EcoT14I; a mixture of EcoT14I and BglII; BstPI; or HindIII. The results are shown in FIG. 5.

As shown in FIG. 5, it is found that a broad size range of fragmented DNAs are produced, so that fragmentation with conversion to a given size cannot be achieved.

When a library was prepared from these fragmented DNAs by the same procedures as in Example 1, the amplification patterns of the fragments obtained were found to show a band intensity different from the band intensity corresponding to the restriction enzyme cleavage pattern of the genomic DNA. It is therefore found that the fragmented DNA does not reflect the copy number and the like in the genomic DNA.

### Example 7

A kit comprising the following reagents was constructed.

(1) T4 DNA ligase,

(2) BAL31 nuclease and DNA Blunting Kit (manufactured by Takara Shuzo Co., Ltd.),

(3) TaKaRa Ex Taq™ DNA polymerase,

(4) EcoRI-NotI-BamHI adapter,

(5) dNTPs mix, reaction buffer for 10×TaKaRa Ex Taq™ DNA polymerase, sterilized water, and

(6) the ER1 primer of SEQ ID NO: 1.

Using the above-mentioned kit and the fragmented DNA of Example 1, the genomic DNA immortalized library of the present invention was prepared. As a result, it was confirmed that a genomic DNA immortalized library which keeps the genetic patterns of the original genomic DNA as in Example 1 can be prepared.

### Equivalent

The invention may be embodied in other specific forms without departing from the spirit or essential characteristics thereof. The present embodiment is therefore to be considered in all respects as illustrative and not restrictive, the scope of the invention being indicated by the appended claims rather than by the foregoing description and all changes which come within the meaning and range of equivalency of the claims are therefore intended to be embraced therein.

### Sequence Listing Free Text

SEQ ID NO: 1 is a sequence for ER1 primer.

SEQ ID NO: 2 is a sequence of a primer for amplifying APC gene.

SEQ ID NO: 3 is a sequence of a primer for amplifying APC gene.

SEQ ID NO: 4 is a sequence of a primer for amplifying APC gene.

SEQ ID NO: 5 is a sequence of a primer for amplifying APC gene.

SEQ ID NO: 6 is a sequence of a primer for amplifying APC gene.

SEQ ID NO: 7 is a sequence of a primer for amplifying APC gene.

SEQ ID NO: 8 is a sequence of a primer for amplifying APC gene.

SEQ ID NO: 9 is a sequence of a primer for amplifying APC gene.

SEQ ID NO: 10 is a sequence of a primer for amplifying APC gene.

SEQ ID NO: 11 is a sequence of a primer for amplifying APC gene.

SEQ ID NO: 12 is a sequence of a primer for amplifying APC gene.

SEQ ID NO: 13 is a sequence of a primer for amplifying APC gene.

SEQ ID NO: 14 is a sequence of a primer for amplifying APC gene.

SEQ ID NO: 15 is a sequence of a primer for amplifying APC gene.

SEQ ID NO: 16 is a sequence of a primer for amplifying APC gene.

SEQ ID NO: 17 is a sequence of a primer for amplifying APC gene.

SEQ ID NO: 18 is a sequence of a primer for amplifying APC gene.

SEQ ID NO: 19 is a sequence of a primer for amplifying APC gene.

SEQ ID NO: 20 is a sequence of a primer for amplifying cyclin D1 gene.

SEQ ID NO: 21 is a sequence of a primer for amplifying CAB1 gene.

SEQ ID NO: 22 is a sequence of a primer for amplifying cyclin E1 gene.

SEQ ID NO: 23 is a sequence of a primer for amplifying cyclin E1 gene.

SEQ ID NO: 24 is a sequence of a primer for amplifying cyclin E1 gene.

SEQ ID NO: 25 is a sequence of a primer for amplifying cyclin E1 gene.

SEQ ID NO: 26 is a sequence of a primer for amplifying p16 gene.

SEQ ID NO: 27 is a sequence of a primer for amplifying p16 gene.

SEQ ID NO: 28 is a sequence of a primer for amplifying BRCA1 gene.

SEQ ID NO: 29 is a sequence of a primer for amplifying BRCA1 gene.

SEQ ID NO: 30 is a sequence of a primer for amplifying BRCA1 gene.

SEQ ID NO: 31 is a sequence of a primer for amplifying BRCA1 gene.

SEQ ID NO: 32 is a sequence of a primer for amplifying BRCA1 gene.

SEQ ID NO: 33 is a sequence of a primer for amplifying BRCA1 gene.

SEQ ID NO: 34 is a sequence of a primer for amplifying BRCA1 gene.

SEQ ID NO: 35 is a sequence of a primer for amplifying BRCA1 gene.

SEQ ID NO: 36 is a sequence of a primer for amplifying BRCA1 gene.

SEQ ID NO: 37 is a sequence of a primer for amplifying BRCA1 gene.

SEQ ID NO: 38 is a sequence of a primer for amplifying BRCA1 gene.

SEQ ID NO: 39 is a sequence of a primer for amplifying BRCA1 gene.

SEQ ID NO: 40 is a sequence of a primer for amplifying BRCA1 gene.

SEQ ID NO: 41 is a sequence of a primer for amplifying BRCA1 gene.

SEQ ID NO: 42 is a sequence of a primer for amplifying BRCA1 gene.

SEQ ID NO: 43 is a sequence of a primer for amplifying BRCA1 gene.

SEQ ID NO: 44 is a sequence of a primer for amplifying BRCA1 gene.

SEQ ID NO: 45 is a sequence of a primer for amplifying BRCA1 gene.

SEQ ID NO: 46 is a sequence of a primer for amplifying BRCA1 gene.

SEQ ID NO: 47 is a sequence of a primer for amplifying BRCA1 gene.

SEQ ID NO: 48 is a sequence of a primer for amplifying BRCA1 gene.

SEQ ID NO: 49 is a sequence of a primer for amplifying BRCA1 gene.

SEQ ID NO: 50 is a sequence of a primer for amplifying BRCA1 gene.

SEQ ID NO: 51 is a sequence of a primer for amplifying BRCA1 gene.

SEQ ID NO: 52 is a sequence of a primer for amplifying BRCA1 gene.

SEQ ID NO: 53 is a sequence of a primer for amplifying BRCA1 gene.

SEQ ID NO: 54 is a sequence of a primer for amplifying BRCA1 gene.

SEQ ID NO: 55 is a sequence of a primer for amplifying BRCA1 gene.

SEQ ID NO: 56 is a sequence of a primer for amplifying BRCA1 gene.

SEQ ID NO: 57 is a sequence of a primer for amplifying BRCA1 gene.

SEQ ID NO: 58 is a sequence of a primer for amplifying TDG gene.

SEQ ID NO: 59 is a sequence of a primer for amplifying TDG gene.

SEQ ID NO: 60 is a sequence of a primer for amplifying TDG gene.

SEQ ID NO: 61 is a sequence of a primer for amplifying TDG gene.

SEQ ID NO: 62 is a sequence of a primer for amplifying p53 gene.

SEQ ID NO: 63 is a sequence of a primer for amplifying p53 gene.

SEQ ID NO: 64 is a sequence of a primer for amplifying CD59 gene.

SEQ ID NO: 65 is a sequence of a primer for amplifying CD59 gene.

SEQ ID NO: 66 is a sequence of a primer for amplifying topoisomerase I gene.

SEQ ID NO: 67 is a sequence of a primer for amplifying topoisomerase I gene.

SEQ ID NO: 68 is a sequence of a primer for amplifying ATP dependent DNA helicase gene.

SEQ ID NO: 69 is a sequence of a primer for amplifying ATP dependent DNA helicase gene.

What is claimed is:

1. A genomic DNA library maintaining substantially copy numbers of a set of genes or sequences on a genomic DNA and an abundance ratio of said set of genes or sequences on the genomic DNA.

2. The genomic DNA library according to claim 1, which is obtained by carrying out a process comprising the steps of
(1) subjecting a genomic DNA to DNA fragmentation means for generating a mixture of fragmented DNAs having distribution ratio of 1 to 5 as defined by the size ratio (distribution ratio) of the maximum size of fragmented DNA to the minimum size of fragmented DNA and having a size convergence rate of 80% or more, thereby giving a mixture of fragmented DNAs; and
(2) subjecting the mixture of fragmented DNAs obtained in step (1) to nucleic acid amplification, thereby producing DNAs corresponding to said mixture of fragmented DNAs.

3. The genomic DNA library according to claim 2, wherein said mixture of fragmented DNAs obtained in step (1) is a mixture of DNAs having distribution ratio of 1 to 5 as defined by the size ratio (distribution ratio) of the maximum size of fragmented DNA to the minimum size of fragmented DNA.

4. The genomic DNA library according to claim 2, wherein said mixture of fragmented DNAs obtained in step (1) is a mixture of DNAs having a size convergence rate of 80% or more.

5. The genomic DNA library according to claim 2, wherein said mixture of fragmented DNAs obtained in step (1) is a mixture of DNAs having an average size of from 0.8 kbp to 1.5 kbp.

6. The genomic DNA library according to claim 2, wherein said nucleic acid amplification is Polymerase Chain Reaction (PCR) method.

7. A method for producing a genomic DNA library, comprising the steps of
(1) subjecting a genomic DNA to DNA fragmentation means for generating a mixture of fragmented DNAs having distribution ratio of 1 to 5 as defined by the size ratio (distribution ratio) of the maximum size of fragmented DNA to the minimum size of fragmented DNA and having a size convergence rate of 80% or more, thereby giving a mixture of fragmented DNAs; and
(2) subjecting the mixture of fragmented DNAs obtained in step (1) to nucleic acid amplification, thereby producing DNAs corresponding to said mixture of fragmented DNAs, to give a genomic DNA library maintaining substantially copy numbers of a set of genes or sequences on a genomic DNA and an abundance ratio of said set of genes or sequences on the genomic DNA.

8. The method according to claim 7, wherein said DNA fragmentation means is physical means.

9. The method according to claim 8, wherein said physical means is hydrodynamic point-sink shearing method.

10. The method according to claim 7, wherein said mixture of fragmented DNAs is a mixture of DNAs having distribution ratio of 1 to 5 as defined by the size ratio (distribution ratio) of the maximum size of fragmented DNA to the minimum size of fragmented DNA.

11. The method according to claim 7, wherein said mixture of fragmented DNAs is a mixture of DNAs having a size convergence rate of 80% or more.

12. The method according to claim 7, wherein said mixture of fragmented DNAs is a mixture of DNAs having an average size of from 0.8 kbp to 1.5 kbp.

13. The method according to claim 7, comprising the steps of:
(a) subjecting a genomic DNA to said DNA fragmentation means, thereby giving fragmented DNAs;
(b) ligating adapter DNA to the fragmented DNAs obtained in step (a), thereby giving DNA fragments; and
(c) carrying out nucleic acid amplification using the DNA fragments obtained in step (b) as a template and amplification primers, to give a genomic DNA library.

14. The method according to claim 13, wherein said DNA fragmentation means in step (a) is hydrodynamic point-sink shearing method.

15. The method according to claim 13, wherein said nucleic acid amplification in step (c) is Polymerase Chain Reaction (PCR) method.

16. The method according to claim 13, wherein said amplification primers used in the nucleic acid amplification in step (c) are primers selected from the group consisting of:
(i) oligonucleotides having a sequence complementary to said adapter DNA, and
(ii) oligonucleotides further comprising recognition sequences for restriction endonucleases, linker sequences and promoter sequence for RNA polymerase, in the sequence of the oligonucleotides of the above item (i).

17. The method according to claim 13, wherein the nucleic acid amplification in step (c) is carried out by using a DNA polymerase having a proofreading activity.

18. The method according to claim 17, wherein said DNA polymerase is a thermostable DNA polymerase.

19. The method according to claim 17, wherein said DNA polymerase is a mixture of a DNA polymerase having 3′→5′ exonuclease activity and a DNA polymerase having no 3′→5′ exonuclease activity.

20. The method according to claim 17, wherein said DNA polymerase is a mixture of at least two kinds of DNA polymerases, each having 3′→5′ exonuclease activity.

21. The method according to claim 17, wherein said DNA polymerase is a mixture of α type DNA polymerase and non-α, non-pol I type DNA polymerase.

22. A kit for producing a genomic DNA library, comprising the following amplification reagents (1) to (6):
(1) DNA ligase,
(2) enzymes for blunting a terminal of DNA,
(3) thermostable DNA polymerase,
(4) adapter DNA,
(5) reagents for PCR, and
(6) amplification primers selected from the group consisting of:
(i) oligonucleotides each having a sequence complementary to said adapter DNA, and
(ii) oligonucleotides further comprising at least one selected from the group consisting of recognition sequences for restriction endonucleases, linker sequences and promoter sequence for RNA polymerase, in the sequence of the oligonucleotides of the above item (i), and
comprising an instruction manual showing a procedure for carrying out the method of claim 7 by using said amplification reagents,
wherein the kit is used for production of the genomic DNA library of any one of claims 1 to 6.


SEQUENCE LISTING (69 sequences)

SEQ ID NO: 1 - DNA, 22 bp, Artificial Sequence
  Description of Artificial Sequence a sequence for ER1 primer
ggaattcggcggccgcggatcc

SEQ ID NO: 2 - DNA, 21 bp, Artificial Sequence
  Description of Artificial Sequence a sequence of a primer for amplifying APC gene
caactctaattagatgaccca

SEQ ID NO: 3 - DNA, 21 bp, Artificial Sequence
  Description of Artificial Sequence a sequence of a primer for amplifying APC gene
gagagtatgaattctgtactt

SEQ ID NO: 4 - DNA, 20 bp, Artificial Sequence
  Description of Artificial Sequence a sequence of a primer for amplifying APC gene
cagacttattgtgtagaaga

SEQ ID NO: 5 - DNA, 20 bp, Artificial Sequence
  Description of Artificial Sequence a sequence of a primer for amplifying APC gene
ctcctgaagaaaattcaaca

SEQ ID NO: 6 - DNA, 20 bp, Artificial Sequence
  Description of Artificial Sequence a sequence of a primer for amplifying APC gene
actccagatggattttcttg

SEQ ID NO: 7 - DNA, 20 bp, Artificial Sequence
  Description of Artificial Sequence a sequence of a primer for amplifying APC gene
ggctggcttttttgctttac

SEQ ID NO: 8 - DNA, 23 bp, Artificial Sequence
  Description of Artificial Sequence a sequence of a primer for amplifying APC gene
gtcgtaattttgtttctaaactc

SEQ ID NO: 9 - DNA, 21 bp, Artificial Sequence
  Description of Artificial Sequence a sequence of a primer for amplifying APC gene
tgaaggactcggatttcacgc

SEQ ID NO: 10 - DNA, 23 bp, Artificial Sequence
  Description of Artificial Sequence a sequence of a primer for amplifying APC gene
atttgaatactacagtgttaccc

SEQ ID NO: 11 - DNA, 24 bp, Artificial Sequence
  Description of Artificial Sequence a sequence of a primer for amplifying APC gene
cttgtattctaatttggcataagg

SEQ ID NO: 12 - DNA, 22 bp, Artificial Sequence
  Description of Artificial Sequence a sequence of a primer for amplifying APC gene
gttactgcatacacattgtgac

SEQ ID NO: 13 - DNA, 23 bp, Artificial Sequence
  Description of Artificial Sequence a sequence of a primer for amplifying APC gene
acttctatctttttcagaacgag

SEQ ID NO: 14 - DNA, 23 bp, Artificial Sequence
  Description of Artificial Sequence a sequence of a primer for amplifying APC gene
atttgaatactacagtgttaccc

SEQ ID NO: 15 - DNA, 26 bp, Artificial Sequence
  Description of Artificial Sequence a sequence of a primer for amplifying APC gene
gtttctcttcattatattttatgcta

SEQ ID NO: 16 - DNA, 23 bp, Artificial Sequence
  Description of Artificial Sequence a sequence of a primer for amplifying APC gene
aagcctaccaattatagtgaacg

SEQ ID NO: 17 - DNA, 20 bp, Artificial Sequence
  Description of Artificial Sequence a sequence of a primer for amplifying APC gene
ggctggcttttttgctttac

SEQ ID NO: 18 - DNA, 20 bp, Artificial Sequence
  Description of Artificial Sequence a sequence of a primer for amplifying APC gene
ctgccatgccaacaaagtca

SEQ ID NO: 19 - DNA, 21 bp, Artificial Sequence
  Description of Artificial Sequence a sequence of a primer for amplifying APC gene
cttttttggcattgcggagct

SEQ ID NO: 20 - DNA, 20 bp, Artificial Sequence
  Description of Artificial Sequence a sequence of a primer for amplifying cyclin D1 gene
atgagcaagctgcccaggga

SEQ ID NO: 21 - DNA, 20 bp, Artificial Sequence
  Description of Artificial Sequence a sequence o a primer for amplifying CAB1 gene
tcacgcccgggcccccagct

SEQ ID NO: 22 - DNA, 20 bp, Artificial Sequence
  Description of Artificial Sequence a sequence of a primer for amplifying cyclin E1 gene
atggaacaccagctcctgtg

SEQ ID NO: 23 - DNA, 20 bp, Artificial Sequence
  Description of Artificial Sequence a sequence of a primer for amplifying cyclin E1 gene
tcagatgtccacgtcccgca

SEQ ID NO: 24 - DNA, 20 bp, Artificial Sequence
  Description of Artificial Sequence a sequence of a primer for amplifying cyclin E1 gene
gtgctcacccggcccggtgc

SEQ ID NO: 25 - DNA, 20 bp, Artificial Sequence
  Description of Artificial Sequence a sequence of a primer for amplifying cyclin E1 gene
ggagagggctgccccctgcc

SEQ ID NO: 26 - DNA, 20 bp, Artificial Sequence
  Description of Artificial Sequence a sequence of a primer for amplifying p16 gene
atggagccggcggcggggag

SEQ ID NO: 27 - DNA, 20 bp, Artificial Sequence
  Description of Artificial Sequence a sequence of a primer for amplifying p16 gene
tcaatcggggatgtctgagg

SEQ ID NO: 28 - DNA, 22 bp, Artificial Sequence
  Description of Artificial Sequence a sequence of a primer for amplifying BRCA1 gene
acctgccacagtagatgctcag

SEQ ID NO: 29 - DNA, 22 bp, Artificial Sequence
  Description of Artificial Sequence a sequence of a primer for amplifying BRCA1 gene
actgcacatacatccctgaacc

SEQ ID NO: 30 - DNA, 22 bp, Artificial Sequence
  Description of Artificial Sequence a sequence of a primer for amplifying BRCA1 gene
ggagagagcagctttcactaac

SEQ ID NO: 31 - DNA, 23 bp, Artificial Sequence
  Description of Artificial Sequence a sequence o a primer for amplifying BRCA1 gene
ccataccacgacatttgacagag

SEQ ID NO: 32 - DNA, 22 bp, Artificial Sequence
  Description of Artificial Sequence a sequence of a primer for amplifying BRCA1 gene
taggtgtggtttctgcataggg

SEQ ID NO: 33 - DNA, 24 bp, Artificial Sequence
  Description of Artificial Sequence a sequence of a primer for amplifying BRCA1 gene
gtccgcctatcattacatgtttcc

SEQ ID NO: 34 - DNA, 22 bp, Artificial Sequence
  Description of Artificial Sequence a sequence of a primer for amplifying BRCA1 gene
aacaccactgagaagcgtgcag

SEQ ID NO: 35 - DNA, 22 bp, Artificial Sequence
  Description of Artificial Sequence a sequence of a primer for amplifying BRCA1 gene
ccatcatgtgagtcatcagaac

SEQ ID NO: 36 - DNA, 23 bp, Artificial Sequence
  Description of Artificial Sequence a sequence of a primer for amplifying BRCA1 gene
caacataacagatgggctggaag

SEQ ID NO: 37 - DNA, 22 bp, Artificial Sequence
  Description of Artificial Sequence a sequence of a primer for amplifying BRCA1 gene
aggcttgccttcttccgatagg

SEQ ID NO: 38 - DNA, 24 bp, Artificial Sequence
  Description of Artificial Sequence a sequence of a primer for amplifying BRCA1 gene
ggcatcatacatgttagctgactg

SEQ ID NO: 39 - DNA, 22 bp, Artificial Sequence
  Description of Artificial Sequence a sequence of a primer for amplifying BRCA1 gene
tcttcaaggtgggaactgcgtc

SEQ ID NO: 40 - DNA, 23 bp, Artificial Sequence
  Description of Artificial Sequence a sequence of a primer for amplifying BRCA1 gene
ctcgttactggaagttagcactc

SEQ ID NO: 41 - DNA, 22 bp, Artificial Sequence
  Description of Artificial Sequence a sequence o a primer for amplifying BRCA1 gene
aaccacaggaaagcctgcagtg

SEQ ID NO: 42 - DNA, 24 bp, Artificial Sequence
  Description of Artificial Sequence a sequence of a primer for amplifying BRCA1 gene
ttggctctttctgtccctcccatc

SEQ ID NO: 43 - DNA, 22 bp, Artificial Sequence
  Description of Artificial Sequence a sequence of a primer for amplifying BRCA1 gene
ttcacaacgccttacgcctctc

SEQ ID NO: 44 - DNA, 22 bp, Artificial Sequence
  Description of Artificial Sequence a sequence of a primer for amplifying BRCA1 gene
aggacacgtgtagaacgtgcag

SEQ ID NO: 45 - DNA, 22 bp, Artificial Sequence
  Description of Artificial Sequence a sequence of a primer for amplifying BRCA1 gene
tcctaatctcgtgatctgcccg

SEQ ID NO: 46 - DNA, 22 bp, Artificial Sequence
  Description of Artificial Sequence a sequence of a primer for amplifying BRCA1 gene
agcctctgattctgtcaccagg

SEQ ID NO: 47 - DNA, 23 bp, Artificial Sequence
  Description of Artificial Sequence a sequence of a primer for amplifying BRCA1 gene
cagcatcaccagcttatctgaac

SEQ ID NO: 48 - DNA, 22 bp, Artificial Sequence
  Description of Artificial Sequence a sequence of a primer for amplifying BRCA1 gene
gaactggaatatgccttgaggg

SEQ ID NO: 49 - DNA, 22 bp, Artificial Sequence
  Description of Artificial Sequence a sequence of a primer for amplifying BRCA1 gene
tctcaatggcgcaaatggatcc

SEQ ID NO: 50 - DNA, 22 bp, Artificial Sequence
  Description of Artificial Sequence a sequence of a primer for amplifying BRCA1 gene
ccaaagtgctaggattacaggg

SEQ ID NO: 51 - DNA, 24 bp, Artificial Sequence
  Description of Artificial Sequence a sequence o a primer for amplifying BRCA1 gene
cctgtgtgaaagtatctagcactg

SEQ ID NO: 52 - DNA, 24 bp, Artificial Sequence
  Description of Artificial Sequence a sequence of a primer for amplifying BRCA1 gene
cagaaatcatcaggtggtgaacag

SEQ ID NO: 53 - DNA, 22 bp, Artificial Sequence
  Description of Artificial Sequence a sequence of a primer for amplifying BRCA1 gene
accttcatgctcttgagaaggg

SEQ ID NO: 54 - DNA, 23 bp, Artificial Sequence
  Description of Artificial Sequence a sequence of a primer for amplifying BRCA1 gene
gagacagactctcccattgagag

SEQ ID NO: 55 - DNA, 22 bp, Artificial Sequence
  Description of Artificial Sequence a sequence of a primer for amplifying BRCA1 gene
atgtgggcagagaagacttctg

SEQ ID NO: 56 - DNA, 22 bp, Artificial Sequence
  Description of Artificial Sequence a sequence of a primer for amplifying BRCA1 gene
attgcgccatcacactctagcc

SEQ ID NO: 57 - DNA, 22 bp, Artificial Sequence
  Description of Artificial Sequence a sequence of a primer for amplifying BRCA1 gene
acccttgcatagccagaagtcc

SEQ ID NO: 58 - DNA, 24 bp, Artificial Sequence
  Description of Artificial Sequence a sequence of a primer for amplifying TDG gene
agcatggctttcttcttcctgttc

SEQ ID NO: 59 - DNA, 24 bp, Artificial Sequence
  Description of Artificial Sequence a sequence of a primer for amplifying TDG gene
cagacacagaaactctctgctatg

SEQ ID NO: 60 - DNA, 22 bp, Artificial Sequence
  Description of Artificial Sequence a sequence of a primer for amplifying TDG gene
caccatatgctgcctcataacc

SEQ ID NO: 61 - DNA, 22 bp, Artificial Sequence
  Description of Artificial Sequence a sequence o a primer for amplifying TDG gene
aacatggtggaaaggaccacgc

SEQ ID NO: 62 - DNA, 22 bp, Artificial Sequence
  Description of Artificial Sequence a sequence of a primer for amplifying p53 gene
atcctggctaacggtgaaaccc

SEQ ID NO: 63 - DNA, 24 bp, Artificial Sequence
  Description of Artificial Sequence a sequence of a primer for amplifying p53 gene
tgatgagaggtggatgggtagtag

SEQ ID NO: 64 - DNA, 19 bp, Artificial Sequence
  Description of Artificial Sequence a sequence of a primer for amplifying CD59 gene
tctcacatggaacgctttc

SEQ ID NO: 65 - DNA, 21 bp, Artificial Sequence
  Description of Artificial Sequence a sequence of a primer for amplifying CD59 gene
taaatacagccaagatcataa

SEQ ID NO: 66 - DNA, 20 bp, Artificial Sequence
  Description of Artificial Sequence a sequence of a primer for amplifying topoisomerase I gene
ggaatttgtcagcgttctac

SEQ ID NO: 67 - DNA, 21 bp, Artificial Sequence
  Description of Artificial Sequence a sequence of a primer for amplifying topoisomerase I gene
caatgcctgtaaaactaatga

SEQ ID NO: 68 - DNA, 20 bp, Artificial Sequence
  Description of Artificial Sequence a sequence of a primer for amplifying ATP dependent DNA helicase gene
ccctccctgttcgtgtaccc

SEQ ID NO: 69 - DNA, 21 bp, Artificial Sequence
  Description of Artificial Sequence a sequence of a primer for amplifying ATP dependent DNA helicase gene
agaccactcttcagcccgtaa